(19) United States
(12) Patent Application Publication
(10) Pub. No.: US 2006/0147935 A1
(43) Pub. Date: Jul. 6, 2006

(54) Methods and means for nucleic acid sequencing

(76) Inventor: Sten Linnarsson, STOCKHOLM (SE)

Correspondence Address: NIXON & VANDERHYE, PC, 901 NORTH GLEBE ROAD, 11TH FLOOR, ARLINGTON, VA, 22203, US

(21) Appl. No.: 10/544,987

(22) PCT Filed: Feb. 9, 2004
(86) PCT No.: PCT/IB04/00803

§ 371 Date: Sep. 23, 2005

Related U.S. Application Data
(60) Provisional application No. 60/446,553, filed on Feb. 12, 2003.

(30) Foreign Application Priority Data
Feb. 12, 2003 (GB) 0303191.1

(51) Int. Cl. C12Q 1/68; G06F 19/00

(52) U.S. Cl. 435/6; 702/20

(57) ABSTRACT

Nucleic acid sequencing-by-synthesis. Primed synthesis of a second strand complementary to a template strand in repeated sets of steps, each step comprising providing one or more of the possible nucleotide complementarity classes for incorporation into the synthesized strand, and each set of steps comprising providing all four possible nucleotide complementarity classes. Three of the four possible nucleotide complementarity classes may first be provided for incorporation into the synthesized strand, then separately the fourth nucleotide complementarity class alone. Also, a DNA molecule consisting of a stem portion and first and second loop portions, wherein the stem portion consists of a first strand and a second strand, wherein the first strand and second strand are equal in length, complementary and annealed together, wherein the first loop portion joins the 3′ end of the first strand to the 5′ end of the second strand and the second loop portion joins the 3′ end of the second strand to the 5′ end of the first strand so the DNA molecule has no free 5′ or 3′ ends, and uses thereof, especially in sequencing.

The present invention relates to nucleic acid sequencing. The present invention especially relates to “sequencing-by-synthesis” (SBS), in which a nucleic acid strand with a free 3′ end is annealed to nucleic acid containing a template for which sequence information is desired and used to prime second-strand synthesis with determination of nucleotide incorporation providing sequence information. The invention is based in part on an elegant concept that allows for use of unblocked nucleotides in what is termed “chroma sequencing”, overcoming various problems with existing sequencing techniques and allowing for a very large amount of sequence to be obtained in a single day using standard reagents and apparatus. Preferred embodiments allow additional advantages to be achieved. The invention also relates to algorithms and techniques for sequence analysis, and apparatus and systems for sequencing. The present invention allows for automation of a vast sequencing effort, using only standard bench-top equipment that is readily available in the art.

The invention involves primed synthesis of a second strand complementary to a template strand in repeated sets of steps, each step comprising providing one or more but optionally less than all of the possible nucleotide complementarity classes for incorporation into the synthesized strand, and each set of steps comprising providing all four possible nucleotide complementarity classes, optionally in two or more steps, where at least one step comprises adding more than one nucleotide complementarity class. Preferably, this involves first providing three of the four possible nucleotide complementarity classes for incorporation into the synthesized strand, then separately providing the fourth nucleotide complementarity class alone. Strand elongation stops with the last step of nucleotide incorporation, e.g. on provision of the fourth nucleotide, as other nucleotides are not present. Determination of the number and optionally the kind of nucleotides between the stops allows for rapid determination of information about base composition and/or sequence of the template. Where a single “stopping nucleotide” is used at a time, performance of four runs using each of the four different nucleotides to stop elongation provides information that can be used to determine very rapidly and easily the complete template sequence.

Although many different methods are used in genomic research, direct sequencing is by far the most valuable. In fact, if sequencing could be made efficient enough, then all three of the major scientific questions in genomics (sequence determination, genotyping, and gene expression analysis) could be addressed. A model species could be sequenced, individuals could be genotyped by whole-genome sequencing and RNA populations could be exhaustively analyzed by conversion to cDNA and sequencing (counting the number of copies of each mRNA directly).

Other examples of scientific and medical problems that can be addressed by sequencing include epigenomics (the study of methylated cytosines in the genome—by bisulfite conversion of unmethylated cytosine to uridine and then comparing the resulting sequence to an unconverted template sequence), protein-protein interactions (by sequencing hits obtained in a yeast two-hybrid experiment), protein DNA interactions (by sequencing DNA fragments obtained after chromosome immunoprecipitation) and many other. Thus, highly efficient methods for DNA sequencing are desirable.

But in order to replace auxiliary methods such as microarrays and PCR fragment analysis, very high sequencing throughput is required. For example, a living cell contains about 300,000 copies of messenger RNA, each about 2,000 bases long on average. Thus to completely sequence the RNA in even a single cell, 600 million nucleotides must be probed. In a complex tissue composed of dozens of different cell types, the task becomes even more difficult as cell-type specific transcripts are further diluted. Gigabase daily throughput will be required to meet these demands. The table below shows some estimates on the throughput required for each experiment (humans, unless indicated otherwise):

| Experiment | Throughput required | |
|---|---|---|
| Genome sequence (10× de novo) | 30 | Gbp |
| Whole-genome polymorphisms | 3 | Gbp |
| Complete haplotype map (200 individuals) | 600 | Gbp |
| Gene expression | 600 | Mbp |
| Epigenomics | 3 | Gbp |
| Ten million protein interactions | 400 | Mbp |
| Entire biosphere (one species per genus) | ˜300 | Tbp |

The present invention place all of the above within reach at reasonable cost.

Methods for DNA Sequencing

Sanger sequencing (Sanger et al. PNAS 74 no. 12: 5463-5467, 1977) using fluorescent dideoxy nucleotides is the most widely used method, and has been successfully automated in 96 and even 384-capillary sequencers. However, the method relies on the physical separation of a large number of fragments corresponding to each base position of the template and is thus not readily scalable to ultra-high throughput sequencing (the best current instruments generate ˜2 million nucleotides of sequence per day).

Sequences can also be obtained indirectly by probing a target polynucleotide with probes selected from a panel of probes.

Sequencing-by-hybridization uses a panel of probes representing all possible sequences up to a certain length (i.e. a set of all k-mers, where k is limited by the number of probes that can fit on the microarray surface; with one million probes, k=10 can be used) and hybridizes the template. Reconstructing the template sequence from the set of probes is complicated and made more difficult by the inherently unpredictable nature of hybridization kinetics and the combinatorial explosion of the number of probes required to sequence larger templates. Even if these problems can be overcome, the throughput will necessarily be low, as one microarray carrying millions of probes is required for each template and the arrays are not usually reusable.

Nanopore sequencing (US Genomics, U.S. Pat. No. 6,355,420) uses the fact that as a long DNA molecule is forced through a nanopore separating two reaction chambers, bound probes can be detected as changes in the conductance between the chambers. By decorating DNA with a subset of all possible k-mers, it is possible to deduce a partial sequence. So far, no viable strategy has been proposed for obtaining a full sequence by the nanopore approach, although if it were possible, staggering throughput could in principle be achieved (on the order of one human genome in thirty minutes).

Various approaches have been designed for sequencing by synthesis (SBS).

In order to increase sequencing throughput it would be desirable to be able to visualize the incorporation of each base on a large number of templates in parallel, e.g. on a glass surface or similar reaction chamber. This is achieved by SBS (see e.g. Malamede et al. U.S. Pat. No. 4,863,849, Kumar U.S. Pat. No. 5,908,755). There are two approaches to SBS: either a byproduct released from each incorporated nucleotide is detected, or a permanently attached label is detected.

Pyrosequencing (e.g. WO9323564) determines the sequence of a template by detecting the byproduct of each incorporated monomer in the form of inorganic diphosphate (PPi). In order to keep the reactions of all template molecules synchronized, monomers are added one at a time and unincorporated monomers are degraded before the next addition. However, homopolymeric subsequences (runs of the same monomer) pose a problem as multiple incorporations cannot be prevented. Synchronization eventually breaks down (because lack of incorporation or misincorporation at a small fraction of the templates add up to eventually overwhelm the true signal), and the best current systems can read only about 20-30 bases with a combined throughput of about 200,000 bases/day.

While Sanger sequencing requires an elaborate apparatus (i.e. a capillary) for each template, Pyrosequencing is readily amenable to parallelization in a single reaction chamber. U.S. Pat. No. 6,274,320 describes the use of rolling-circle amplification to produce tandemly repeated linear single-stranded DNA molecules attached to an optic fiber, analyzed in a Pyrosequencing reaction which can then proceed in parallel. In principle, the throughput of such a system is limited only by the surface area (number of template molecules), the reaction speed and the imaging equipment (resolution). However, the need to prevent PPi from diffusing away from the detector before being converted to a detectable signal means that the number of reaction sites must be limited in practice. In U.S. Pat. No. 6,274,320, each reaction is constrained to occur in a miniature reaction vessel located on the tip of an optic fiber, thus limiting the number of sequences to one per fiber.

Even more limiting are the short read lengths achieved by Pyrosequencing (<30 bp). Such short sequences are not directly useful in whole-genome sequencing, and the complex set of balancing reactions make it difficult to extend the read length much further. Only occasionally and for specific templates have read lengths up to 100 bp been reported.

A similar scheme with detection of a released label is described in U.S. Pat. No. 6,255,083. A scheme with sequential addition of nucleotides and detection of a label that is then cleaved off with an exonuclease is described in WO01/23610.

The principal advantage of detecting a released label or byproduct is that the template remains free of label at subsequent steps. However, because the signal diffuses away from the template, it may be difficult to parallellize such sequencing schemes on a solid surface such as a microarray.

Instead of detecting a released byproduct, one can detect each incorporated nucleotide as it is added to the growing polymer. In principle, such a scheme would proceed like pyrosequencing (adding one base at a time, cycling among the four natural nucleotides), but would instead use labeled nucleotide analogs (i.e. fluorescent). As an example, Polony sequencing (Mitra R D, Church G M., Nucleic Acids Res Dec. 15, 1999;27(24):e34 “In situ localized amplification and contact replication of many individual DNA molecules”) is based on sequential addition of fluorescently labeled nucleotides.

Detecting a label attached to each incorporated nucleotide presents an additional difficulty in that signal generated in each step must be removed, computationally subtracted or physically quenched in preparation for the next step. Such removal can be accomplished, e.g. by photobleaching or by using cleavable linkers between the nucleotide and the label. For example, polony sequencing uses specially designed fluorescent nucleotides, which carry a dithiol linker between the nucleotide and the fluorochrome. According to unpublished observations, the linker can be efficiently cleaved using a reducing agent such as dithiothreitol to at least 99.8% pure nucleotide.

Since the read length in SBS methods is primarily limited by the loss of synchrony that occurs in each step, it would be desirable to be able to add all four nucleotides to the sequencing reaction, yet retain the ability to halt the reaction between each incorporation of a base. In that way, all four nucleotides would always be available (thus limiting misincorporation rates), yet it would be possible to monitor each incorporated base.

A number of investigators have independently conceived of a solution sometimes termed base-addition sequencing strategy (BASS). The reaction is prevented from proceeding more than one step at a time by the use of 3′-blocked monomers, but the blocking moiety is labile (e.g. photocleavable or chemically degradable) so that the 3′-OH group can be exposed in preparation for the next synthesis step.

BASS comprises:

1. Providing a single-stranded template and an annealed primer;

2. Adding 3′-OH-blocked fluorescent nucleotides;

3. Adding polymerase, incorporating a single nucleotide;

4. Reading the fluorescence;

5. Removing the blocking group e.g. by photocleavage;

6. Repeating steps 2-5.

Variations on this theme use permanently 3′-OH-blocked nucleotides that are removed using exonuclease (WO1/23610, WO93/21340) or labile 3′-OH-blocked nucleotides that can be restored to functional 3′-OH groups (U.S. Pat. No. 5,302,509, WO00/50642, WO91/06678, WO93/05183).

All of the BASS schemes have the following in common:
Blocked or terminating nucleotides are used to prevent synthesis to proceed more than one step at a time.
The nucleotide incorporated at each step is also labeled, usually with a fluorochrome.
At the end of each cycle, the blocking moiety (or the entire terminal nucleotide) is removed in preparation for the next cycle.

Together, these requirements place formidable demands on the enzymes used in BASS:
They must accept nucleotides simultaneously blocked at their 3′ (where modifications are not usually tolerated by the enzyme) and fluorescently labeled.
They must incorporate such nucleotides efficiently enough so that only a negligible fraction of all templates fall out of synchrony in each cycle.
They must be capable of stringently discriminating base-pairings of such nucleotides.
They must not remove the blocking group or terminating nucleotide prematurely.

The fact that no one has so far been able to get BASS to work suggests that these difficulties are insurmountable. For example, in (Metzker et al. “Termination of DNA synthesis by novel 3′-modified-deoxyribonucleoside 5′-triphosphates”, Nucleic Acids Res 1994: 22(20):4259-67), no enzyme among eight surveyed was capable of tolerating both 3′-blocked dUTP and 3′-blocked dCTP, even without the added complication of a fluorescent label. Thus finding an enzyme that can accept 3′-blocked and fluorescently labeled versions of all four nucleotides seems almost hopeless.

In conclusion, if a sequencing-by-incorporation method could be made to work, then one could conceivably sequence millions of templates attached to a surface in parallel. The major attraction of detecting an incorporated rather than released label is that reactions could be parallellized on a surface. For example, on a 10×10 cm surface such a system could be capable of sequencing e.g. ˜600 000 bp/s on 37-million templates at 60 s per cycle (assuming Poisson distribution of 1 template/10 μm), achieving 50 Gb/24 hours. In principle, ten human genomes could be sequenced every day on such a system. The cost of the system would be comparable to a fluorescence scanner and the running cost would be comparable that of a current Sanger sequencer.

The major remaining obstacles to achieving that goal are: first, that read lengths in SBS are too short to be useful in sequencing large genomes and second, that a reliable way to place templates at sufficiently high density on a surface has not been developed.

The present invention in various aspects ingeniously solves prior art problems.

### BRIEF DESCRIPTION OF THE FIGURES

FIG. 1 illustrates a template (top row, showing the sequenced strand) sequenced with chroma sequencing using each of the natural nucleotides (indicated on the left) as a stopping nucleotide. Each chroma sequence is shown as a series of dashes (measuring the number of intervening bases) and letters (measuring the number of uninterrupted stopping nucleotides). From the figure, it is evident that by lining up the reads, the original sequence can be recovered by reading columns.

FIG. 2

In the nucleotide incorporation assay of example II, the figure shows fluorescence (in arbitrary units) after attempted incorporation of dTTP (labeled in Cy3), DATP and dGTP with and without DNA polymerase (Klenow). The expected outcome is two incorporated dTTP, and the figure clearly demonstrates that enough signal is generated from such an incorporation event to reliably detect the incorporation above background noise.

FIG. 3 illustrates an embodiment of a reaction chamber suitable for solid-phase chroma sequencing in a regular microarray scanner. The illustration shows a chamber assembly using a regular 25×75 mm glass slide (1) to which the templates can be spotted or randomly attached. A rubber gasket (2) seals the glass to the chamber during reactions. Inlet (3) and outlet (4) ports are connected via connectors (5) to a reagent distribution system as illustrated in FIG. 4.

FIG. 4 illustrates an embodiment of a reagent distribution system suitable for performing chroma sequencing in the reaction chamber of FIG. 3. A 10-port valve (1) allows distribution of reagents into and out of the chamber (2) and waste (6), and up to eight reagent vessels (3) can contain the different reagents and wash buffers as required by any given chroma sequencing scheme. The syringe pump (4) and valve (1) can easily be motorized and computer-controlled together with the scanner (5, with partial view shown of slide holder) for a completely automated system.

The present invention is based on development of a novel sequencing strategy that improves on previously described sequencing-by-synthesis methods while allowing for most of their difficulties to be avoided. It is a strategy that is easy to parallelize, that directly visualizes the incorporation of each monomer (i.e. no size fractionation is required) and that provides the possibility for long read lengths.

The invention is based on the realization that in SBS methods, contrary to what has been assumed, it is not necessary to halt at each position (by adding bases one at a time as in pyrosequencing or the method of WO1/23610, or by using blocked nucleotides as in BASS).

Instead, sequencing can proceed in hops, jumping from each occurrence of a particular ‘stopping” nucleotide to the next. The intervening nucleotides may be labeled. The stopping nucleotide may be labeled. This provides an improvement which may be an ideal compromise between schemes where blocking groups are used (in which each step is productive, but de-locking is problematic) and schemes where synchronization is achieved by adding bases one at a time (in which de-blocking is avoided at the cost of making most steps unproductive, exacerbating the loss-of-synchrony problem). Also, compared with the case of BASS, the invention removes the need to put the label on the same nucleotide as the blocking group.

One aspect of the invention provides sequencing-by-synthesis characterized by incorporation of nucleotides in a step-wise manner, wherein a step potentially allows for incorporation of ore than one nucleotide.

In a preferred embodiment one step potentially allows for incorporation of three of the four possible nucleotides, dependent on the underlying template sequence. Preferably a separate step allows for incorporation of the fourth possible nucleotide, i.e. the one remaining other than the three that could potentially be incorporated in the first step.

In other embodiments, different steps are performed to allow in a set of steps incorporation of all four nucleotides, wherein at least one step allows for incorporation of more than one but less than all of the possible nucleotides. As is discussed further below, prior art methods can be summarized either as having four separate repeated steps in a set that can be cycled, each step allowing in principle for incorporation of only one of the four nucleotides (the actual number of nucleotides incorporated depending on the underlying template sequence), or as having a single repeated step comprising all four blocked nucleotides again allowing for incorporation of only one of the four nucleotides in each step, both of which can be summarized as a “1-1-1-1” process single step allowing in principle for incorporation of all four nucleotides, which can be summarized as a “4” process, is not useful for sequencing since the sequenced strand would immediately polymerize to the end of the template. The present invention in different embodiments allows for performance of a method of sequencing-by-synthesis characterized by incorporation of nucleotides in steps that conform to a pattern other than “4” or “1-1-1-1”. Thus, in a preferred embodiment nucleotides are incorporated in a set of steps conforming to “3-1”, as already mentioned. In other embodiments, a set of steps conforms to “2-2” or “1-2-1”, or to an irregular pattern where nucleotides may be repeated within a set of steps (e.g. “2-2-3”). Sets of steps are cycled as desired. Furthermore, combinations of sets of steps with different patterns may be made.

According to one aspect of the present invention there is provided a method of determining sequence and/or base composition information for a nucleic acid, the method comprising:

(i) providing a nucleic acid comprising a first strand that comprises a nucleic acid template, wherein a free 3′ end of a nucleic acid strand annealed to the first strand of the nucleic acid template allows for elongation of a strand of nucleic acid complementary to the nucleic acid template by template sequence-dependent incorporation of nucleotides into the strand of nucleic acid complementary to the nucleic acid template by a template-dependent nucleic acid polymerase;

(ii) performing a set of one or more steps, which set of one or more steps is cycled a desired number of times or performed in combination with other sets of one or more steps to elongate the strand of nucleic acid complementary to the nucleic acid template allowing for information indicative of base composition or sequence of the nucleic acid to be obtained,
wherein a step comprises:
(a) providing, in the presence of:
   the nucleic acid comprising a first strand that comprises a nucleic acid template,
   said free 3′ end of a nucleic acid strand annealed to the first strand of the nucleic acid template, and
   a template-dependent nucleic acid polymerase; nucleotides selected from one, two, three or four nucleotide complementarity classes for template-dependent incorporation by the nucleic acid polymerase of the nucleotides into the strand of nucleic acid complementary to the nucleic acid template, wherein each of said nucleotides is a natural nucleotide or a nucleotide analog capable of template-dependent incorporation by a nucleic acid polymerase into a DNA strand at a free 3′ end of the nucleic acid strand, and within each said nucleotide complementarity class the nucleotides and nucleotide analogs are complementary to one of Adenosine (A), Cytosine (C), Thymine (T) and Guanine (G); and
(b) removing or inactivating unincorporated nucleotides; and
wherein within a set of steps
   nucleotides selected from all four nucleotide complementarity classes are provided and available for template-dependent incorporation,
   in at least one step nucleotides selected from more than one, optionally two, three or four, nucleotide complementarity classes are provided and available for template-dependent incorporation, and the nucleotides in at least one of the nucleotide complementarity classes, if incorporated into the strand of nucleic acid complementary to the nucleic acid template, allow further elongation of the strand of nucleic acid complementary to the nucleic acid template, and
   optionally no nucleotide complementarity class is provided in more than one step, or each nucleotide complementarity class is provided in no more than one of the steps within the set of steps; and
wherein if nucleotides selected from all four complementarity classes are provided in one step then the nucleotides in one, two or three of the nucleotide complementarity classes, if incorporated into the strand of nucleic acid complementary to the nucleic acid template, prevent further elongation of the strand of nucleic acid complementary to the nucleic acid template and all copies present if multiple copies are present;

(iii) performing multiple sets of said steps, cycling sets of steps and/or performing sets of steps in combination with different sets of steps;

(iv) determining the nature of and/or quantity of nucleotides incorporated into the strand of nucleic acid complementary to the nucleic acid template in at least one set of steps by determining the nature and/or quantity of nucleotides incorporated into the strand of nucleic acid complementary to the nucleic acid template in at least one step in each set for which the nature and/or quantity of nucleotides incorporated is determined for the set.

As noted, the invention allows for sequencing without size fractionation.

The free 3′ end of nucleic acid annealed to the first strand 5′ of the nucleic acid (e.g. DNA) template (for which sequence information and/or base composition information is desired), may be provided by a primer (e.g. an oligonucleotide primer) annealed to the first strand, may be provided by a nick in a second strand annealed to the first strand (in which case the portion of the second strand that initially anneals to the nucleic acid template is displaced or degraded during elongation), or may be provided by a self-loop, i.e. a continuation of the first strand that loops back allowing for self-priming.

A nucleotide or nucleotide analog can be defined by its base-pairing properties. All nucleotides or nucleotide analogs that will incorporate complementary to natural adenosine thus belong to the nucleotide complementarity class of thymine, those that incorporate complementary to natural guanine belong to the nucleotide complementarity class of cytosine, those that incorporate complementary to natural thymine belong to the nucleotide complementarity class of adenosine and those that incorporate complementary to natural cytosine belong to the nucleotide complementarity class of guanine. The nucleotide complementarity class thus describes and defines the logical property of a nucleotide or nucleotide analog with respect to template-directed polymerization.

Nucleotides are potentially allowed for incorporation by being provided in the reaction medium, for incorporation by a template-dependent polymerase.

The nucleic acid template may be a deoxyribonucleic acid (DNA), the nucleic acid polymerase may be a DNA-dependent DNA polymerase and the nucleotides may be deoxyribonucleotides or deoxyribonucleotide analogs.

The nucleic acid template may be a deoxyribonucleic acid (DNA), the nucleic acid polymerase may be a DNA-dependent ribonucleic acid (RNA) polymerase and the nucleotides may be ribonucleotides or ribonucleotide analogs.

The nucleic acid template may be a ribonucleic acid (RNA), the nucleic acid polymerase may be a reverse transcriptase and the nucleotides may be deoxyribonucleotides or deoxyribonucleotide analogs.

In preferred embodiments of various aspects of the present invention, nucleotides used in a step in which more than one different nucleotide is potentially incorporated are selected from standard nucleotides.

In some preferred embodiments of various aspects of the present invention, a nucleotide used in a step in which only one of the different nucleotides is potentially incorporated is a nucleotide selected from the standard nucleotides.

In other embodiments, modified nucleotides or analogs may be employed, as discussed further elsewhere herein.

Nucleotides employed in the present invention may be labeled, and labeling may comprise a fluorescent label. Different nucleotides (as between complementarity classes of A, C, G and T) may be labeled with different labels, e.g. different fluorescent labels which may be different colours.

As noted, the invention provides a sequencing-by-synthesis method characterized by incorporation of nucleotides in a scheme other than 4 or 1-1-1-1.

Thus, preferably the incorporation scheme first allows for potential incorporation of 2 or 3 nucleotides, then, generally following a washing step to remove unincorporated nucleotides, in a separate step the incorporation scheme allows for potential incorporation of 2 nucleotides or 1 nucleotide. Combinations of sets of steps may be made to provide an overall reaction scheme.

Of course, appropriate conditions are provided in the reaction medium for performance of template-dependent nucleotide incorporation at the 3′ end of a DNA strand, in accordance with knowledge and techniques available in the art.

In one embodiment, the invention presents a method which comprises a cycle of steps or sets of steps: providing a DNA template, wherein a free 3′ end of a nucleic acid strand annealed to the first strand 5′ of the DNA template (e.g. an annealed primer) allows for synthesis of a DNA strand complementary to the DNA template, adding a set of labeled nucleotides (termed the “intervening” nucleotides) in a first step in the presence of a polymerase under conditions for incorporation of nucleotides into an elongating strand complementary to the template, followed by washing to remove unincorporated nucleotides, then adding a second set of labeled nucleotides (the “stopping” nucleotides) in a second step in the presence of a polymerase under conditions for primer-based incorporation of nucleotides into the elongating strand, followed by washing to remove unincorporated nucleotides, and determining the labels of incorporated nucleotides. The set of steps may be repeated as many cycles or times as desired.

Thus in each step the number (but not the order of) incorporated nucleotides is determined. If the labels for different nucleotides are distinguishable, the number (but not order) of each incorporated nucleotide species will have been determined.

The information on incorporated nucleotides obtained in this way, i.e. by determination of the labels, is called a chroma. A chroma is not a standard DNA sequence, but: It can be used as a signature sequence and aligned to
known DNA sequences;
A set of four (usually) such sequences can be reassembled into a regular DNA sequence (as explained further herein).

Embodiments of the invention, and the concept of a chroma, can be illustrated by reference to a typical sequence obtained by using dA, dC and dG as intervening nucleotides and dT as stopping nucleotide, e.g. written as follows:
dT [1A,2C,1G,1T]-[2A,2C,1G,3T]-[2A,2C,1G,1T]-[0A,1C,0G,1T]
   where the numbers in brackets give the abundances of each intervening nucleotide between each occurrence of dT as measured by their label intensities, plus the number of consecutive dTs.

Several DNA sequences could have generated the data, for example:

ACCGTGCACATTTACAGCTCT
CAGCTCCAAGTTTCACGATCT
etc . . .

A base-calling strategy is provided below that uses the information or chroma obtained from four such sequence reads (using each of the four nucleotides successively as stopping nucleotides) to unambiguously determine the original sequence.

In one aspect, a preferred embodiment of the present invention provides a method (scheme I) comprising:

1. Providing a single-stranded template with an annealed DNA strand with a 3′ end to act as a primer.

2. Adding a set of one or more labeled nucleotides (termed “intervening nucleotides⇄), selected such that at least one nucleotide (termed “stopping nucleotide⇄) complementary to the template is excluded from the set of labeled nucleotides. Usually, three nucleotides carrying distinguishable labels are added (the fourth natural nucleotide being the stopping nucleotide).

3. Optionally adding one or more blocking nucleotides (different from the labeled nucleotides). These are also “stopping nucleotides”. Examples include 3′-O-modified nucleotides, which may carry a photocleavable group that leaves a 3′-OH when illuminated or other modification, acyclic nucleotides and dideoxy nucleotides.

4. Optionally adding one or more nonincorporating inhibitor nucleotides (different from the labeled nucleotides and the blocked nucleotides), which serve to prevent misincorporation at template positions that have no complement in the set of labeled or blocking nucleotides. Examples include 5′-di- and mono-phosphate nucleotides, 5′-(alpha-beta-methylene) triphosphate nucleotides.

5. Incubating with an appropriate polymerase under conditions that cause nucleotides to be added to the growing strand.

6. Washing away unincorporated nucleotides.

7. If any blocking nucleotides were added in step 3
a. Removing blocking moieties, e.g. by photocleavage, enzymatic conversion or chemical reaction.
b. Alternatively, replacing the entire nucleotide by exonuclease treatment and subsequent incorporation of a non-blocked nucleotide (see for example WO1/23610, WO93/21340).

8. Adding the remaining nucleotides (“stopping nucleotides⇄) that are required to ensure that all nucleotides present in the template have had complements added, and incubating with a polymerase (not necessarily the same as in step 5) under conditions that cause nucleotides to be added to the growing strand. The stopping nucleotides may optionally be labeled, and/or 3′-blocked (e.g. as in BASS).

9. Washing away unincorporated nucleotides.

10. Detecting the presence and/or quantity of each labeled nucleotide.

11. Optionally removing or disabling the labels and/or 3′-blocking groups. For example, fluorescent labels may be photobleached.

12. Repeating steps 2-11 until the desired number of cycles have been completed.

Such a sequencing method is particularly suitable for parallelization on a solid phase, both because of its simplicity and because it provides a robust method of synchronization. The scheme can be repeated multiple times by restarting at step 1 with a fresh primer.

Nucleotides added in steps 3 and 8 are referred to as stopping nucleotides, since they prevent (by being blocked or by being absent) polymerization to proceed beyond their complements in step 5. The set of stopping nucleotides can be varied. For example, if the reaction is performed four times from step 1, each of the four natural nucleotides can be used as stopping nucleotide.

A primer anneals by base complementarity to the template, leaving a free 3′ end to which nucleotides can be added one-by-one by a template-dependent DNA polymerase. As noted, a free 3′ end can be generated by nicking one strand of a double-stranded DNA molecule, or by allowing a free 3′ end of a single strand to loop back for self-priming.

Note: a “labeled” molecule shall be taken to include pure labeled molecules as well as mixtures of labeled and unlabeled molecules. For instance, labeled dTTP could be pure fluorescein-labeled dTTP or a mixture of fluorescein-labeled dTTP and regular, unlabeled dTTP. The optimal ratio of labeled to unlabeled is determined by several factors:
The need to obtain enough signal to overcome instrument noise. For example, on a PerkinElmer ScanArray, 2.5 fluorochromes/pixel yield a signal three times the noise level.
The need to avoid having multiple flurochromes in close proximity to avoid fluorescent resonant energy transfer (FRET, which results in one fluorochrome quenching another). FRET decays with the sixth power of the distance, but can still be important over a range of a few nucleotides.
The need to avoid having multiple flurochromes in close proximity to avoid inhibiting the subsequent incorporation of nucleotides by the polymerase (which may be inhibited by steric effects of the bulky fluorochromes).
As another option, one may force the labelled nucleotide fraction to terminate the growing chain, for example by using labelled acyclic or dideoxy nucleotides or by placing the label on or near the 3′-OH. As long as labelled nucleotides make up only a small fraction of all nucleotides, the loss in signal caused by termination remains insignificant, while the loss of synchrony caused by the enzyme's lower affinity for modified nucleotides can be entirely avoided.

Work in the inventor's laboratory has found that ˜2.5% or less of labeled nucleotides works well (see example below). Assuming that the template is 1000 tandem-repeated copies of a 100 bp sequence, at least 25 fluorochromes per template are obtained for each incorporated nucleotide (i.e. >10-fold above noise level on a PerkinElmer ScanArray if each template is within a pixel). Assuming that four nucleotides are incorporated in an average cycle, the labels are spaced on average 1000 bases apart, avoiding both quenching and polymerase inhibition.

In further embodiments of the present invention, scheme I (for example) allows a variant of BASS that relaxes some of the constraints on the polymerase. If the set of intervening nucleotides is labeled but unblocked, while the stopping nucleotide is unlabelled but blocked, then all four nucleotides may be added as a mixture in a single step, then washed and scanned as above. A polymerase that accepts both blocked nucleotides and labeled nucleotides may be used or the labeled intervening nucleotides may be added in a first step and the blocked stopping nucleotide in a second step, using different polymerases. The chroma for such a modified scheme differs in that homopolymers are detected as adjacent cycles with no incorporation; they each terminate with a single stopping nucleotide incorporated, thus scanning the homopolymer stepwise rather than filling it in a single run.

In such a scheme, it may be desirable to use photocleavable fluorochromes (see below) as well as photocleavable 3′-blocking groups. Alternatively, blocking groups removable by mild chemical treatment may be used, for example the allyl group described in Kamal et al. (Tetrahedron Letters 1999, vol. 40, pp. 371-372).

In a particularly simple embodiment, an aspect of the present invention provides a method (scheme II) which comprises:

1. Providing a single-stranded template with a free 3′ end on an annealed DNA strand, to function as a primer.

2. Adding three nucleotides carrying distinguishable labels, e.g. distinguishable fluorescent labels.

3. Optionally adding one or more nonincorporating inhibitor nucleotides (different from the labeled nucleotides). Examples include 5′-di- and mono-phosphate nucleotides, 5′-(alpha-beta-methylene)triphosphate nucleotides.

4. Incubating with an appropriate polymerase under conditions that cause nucleotides to be added to the growing strand.

5. Washing away unincorporated nucleotides.

6. Adding the remaining nucleotide (labeled, e.g fluorescently), and incubating with a polymerase (not necessarily the same as in step 5) under conditions that cause nucleotides to be added to the growing strand.

7. Washing away unincorporated nucleotides.

8. Detecting the presence and quantity of each labeled nucleotide.

9. Disabling the labels (e.g. by photobleaching, not necessarily in every cycle, or by chemical treatment with e.g. dithiothreitol to cleave a disulfide link)

10. Repeating steps 2-7 until the desired number of cycles have been completed.

For example, one may use dA/dG/dC in step 2 (e.g. labeled red/green/blue) and then add dT in step 6 (e.g. labeled yellow). Step 4 will add any number of dA, dG and dC until the first occurrence of a dA in the template, then stop because there is no complementary nucleotide. The fluorescence read in step 8 for dA/dG/dC (e.g. red/green/blue) will be proportional to the number of dA, dG and dC between each dT, whereas the fluorescence for the incorporated dA (e.g. yellow) will be proportional to the number of uninterrupted dTs, and after spectral separation each contribution may be quantified. The sequence obtained can in general be written as a sequence of four numbers giving the number (but not order) of dA, dG, and dC between each dT.

For example, the sequence ACGCTACGCATCAGACTTC (i.e. template TGCGATGCGTAGTCTGAAG) could be written as [1A,2C,1G,1T]-[2A,2C,1G,1T]-[2A,2C,1G,2T]-[0A,1C,0G,0T].

By performing four different reactions according to scheme II, varying the stopping nucleotide among the four possibilities, one can ensure that there is a stop at each different base in one of the four reactions.

Although fluorochromes are convenient to use, not all fluorochromes are easy to bleach. Other kinds of labeling can be used in the above procedure, as long as they can be removed, inactivated or computationally subtracted for each cycle. However, in further embodiments, in order to permit a wider selection of labels, removal (e.g. photobleaching of fluorochromes) can optionally be replaced by full restart, for example as follows:

First, one cycle is performed with labeled, e g. fluorescent, nucleotides. The newly synthesized DNA strand is removed, e.g. by formamide treatment, and a fresh primer is annealed to restart the process. This time, one cycle is performed with unlabeled nucleotides, followed by one cycle with labeled nucleotides. The process is repeated, each time with successively more cycles of unlabeled nucleotides. In this way, only the last cycle in each restart is ever labeled, removing the need to remove the label from previous cycles (e.g. to bleach fluorochromes).

The same approach can also be used to skip over regions that are not of interest, somewhat like moving the read head of a tape recorder.

As an alternative to photobleaching, modified fluorescent nucleotides carrying a cleavable linker between the nucleotide and the fluorochrome can be used. For example, such nucleotides have been described carrying a disulfide bond, which can be efficiently cleaved by a reducing agent such as dithiothreitol (see the work of Rob Mitra and George Church, on polony technology for sequencing and genotyping, findable on the internet using an browser, e.g. http://cbcg.1bl.gov/Genome9/Talks/mitra.pdf, for details including chemical structure. Similarly, Li et al. (PNAS 2003, vol. 100 no. 2, pp. 414-419) describe photocleavable fluorescent nucleotides comprising a photolabile 2-nitrobenzyl linker.

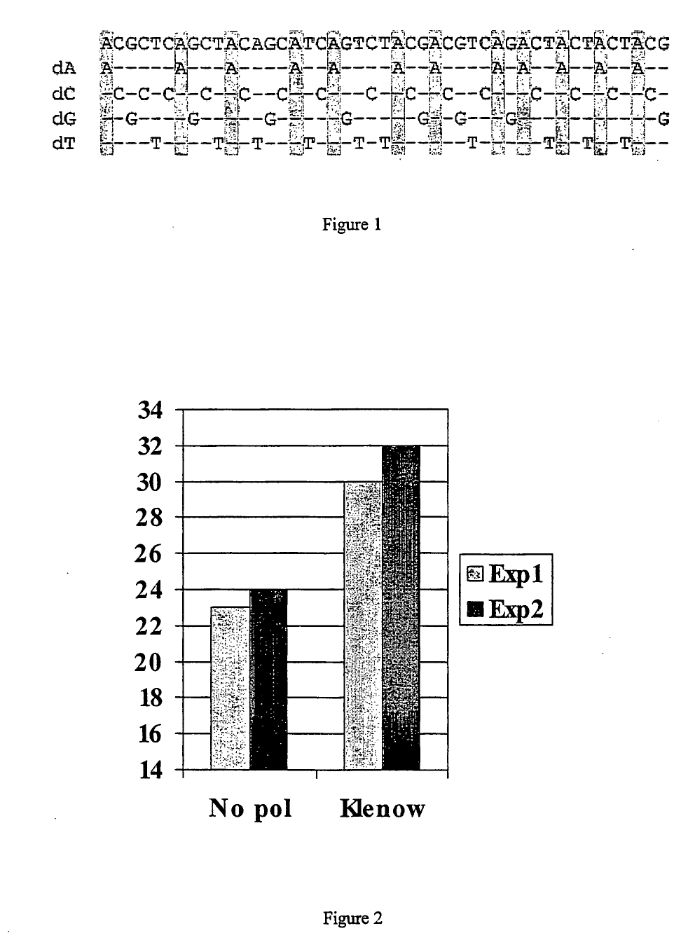

The method according to scheme II allows for achievement of many advantages:
Since one of the four reactions stops at each template position (disregarding homopolymers), the number of cycles required to sequence n bases is n, compared to current SBS methods where most cycles are unproductive (since in such methods one adds a single base at a time, with a <50% chance of being complementary at that position).
Since synthesis is restarted from the primer for each of the four reactions, factors that depend crucially on the number of cycles will be four times less problematic. In particular, loss of synchrony will occur after a number of cycles, but since all templates are effectively resynchronized for each of the four reactions, four times as many bases can be read compared to SBI or Pyrosequencing, under similar conditions (see example below).
Applications that do not need a full sequence (i.e. signature sequencing for gene expression, methyl-cytosine sequencing for epigenomics, as well as SNP analysis for particular SNPs) can use partial sequence obtained from just one of the four reactions. The sequences obtained contain information equivalent to 1 basepair per cycle. See scheme III below. See also FIG. 1 for an illustration of data obtainable for composition of each of dA, dC, dG and dT in separate reactions. Any one of those may be sufficient for the desired purpose, e.g. to determine which of several possible sequences (e.g. with differences in dA nucleotides) is present in a test sample.
Homopolymeric stretches are always measured four times, making them easier to basecall correctly than they would be in SBI or Pyrosequencing. See basecalling algorithm II below.
   Base-Calling Algorithm I (Basic Strategy)

This section of the disclosure sets out exemplary embodiments of aspects of the invention relating to identification of the sequence from the information obtained by means of a method involving use of stopping and intervening nucleotides as disclosed.

By performing four different reactions according to scheme II, varying the stopping nucleotide among the four possibilities, one can ensure that there is a stop at each different base in one of the four reactions. The table below shows the results or chroma that would be obtained from the sequence ACGCTACGCATCAGACTC (template TGCGATGCGTAGTCTGAG) in four cycles using each of the four stopping nucleotides:

| | |
|---|---|
| Stop | Sequence obtained (first four cycles): |

| | |
|---|---|
| dT | [1A,2C,1G,1T] - [2A,2C,1G,1T] - [2A,2C,1G,1T] - [0A,1C,0G,0T] |
| dA | [0C,0G,0T,1A] - [2C,1G,1T,1A] - [2C,1G,0T,1A] - [1C,0G,1T,1A] |
| dG | [1A,1C,0T,1G] - [1A,2C,1T,1G] - [2A,2C,1T,1G] - [1A,2C,1T,0G] |
| dC | [1A,0G,0T,1C] - [0A,1G,0T,1C] - [1A,0G,1T,1C] - [0A,1G,0T,1C] |

Reading from left to right, one can easily see that the first nucleotide must be an A (since the first step for A gives no fluorescence for any of the other bases and hence must have terminated without any intervening nucleotides). Removing the corresponding entry and noting the A yields:

Sequence: A

| | |
|---|---|
| Stop | Sequence obtained: |

| | |
|---|---|
| dT | [1A,2C,1G,1T] - [2A,2C,1G,1T] - [2A,2C,1G,1T] - [0A,1C,0G,0T] |
| dA | [2C,1G,1T,1A] - [2C,1G,0T,1A] - [1C,0G,1T,1A] |
| dG | [1A,1C,0T,1G] - [1A,2C,1T,1G] - [2A,2C,1T,1G] - [1A,2C,1T,0G] |
| dC | [1A,0G,0T,1C] - [0A,1G,0T,1C] - [1A,0G,1T,1C] - [0A,1G,0T,1C] |

Now the only consistent entry on the left side is for C, since it indicates the presence of just one A. Removing the corresponding entry and noting the C we get:

Sequence: AC

| | |
|---|---|
| Stop | Sequence obtained: |

| | |
|---|---|
| dT | [1A,2C,1G,1T] - [2A,2C,1G,1T] - [2A,2C,1G,1T] - [0A,1C,0G,0T] |
| dA | [2C,1G,1T,1A] - [2C,1G,0T,1A] - [1C,0G,1T,1A] |
| dG | [1A,1C,0T,1G] - [1A,2C,1T,1G] - [2A,2C,1T,1G] - [1A,2C,1T,0G] |
| dC | [0A,1G,0T,1C] - [1A,0G,1T,1C] - [0A,1G,0T,1C] |

Now the only consistent entry on the left side is for G:

Sequence: ACG

| | |
|---|---|
| Stop | Sequence obtained: |

| | |
|---|---|
| dT | [1A,2C,1G,1T] - [2A,2C,1G,1T] - [2A,2C,1G,1T] - [0A,1C,0G,0T] |
| dA | [2C,1G,1T,1A] - [2C,1G,0T,1A] - [1C,0G,1T,1A] |
| dG | [1A,2C,1T,1G] - [2A,2C,1T,1G] - [1A,2C,1T,1G] |
| dC | [0A,1G,0T,1C] - [1A,0G,1T,1C] - [0A,1G,0T,1C] |

Now the only consistent entry on the left side is for C, since it indicates just one G between this and the previous C, consistent with the sequence we have so far.

Continuing like this finally provides the entire sequence:
ACGCTACGCATCAGACTC.

In fact, it is easy to see that the sum of fluorescence obtained from intervening nucleotides in each step measures the total distance between each stopping nucleotide, while the fluorescence from the stopping nucleotide measures the number of noninterrupted stopping nucleotides, and that one can therefore always determine the sequence from a set of four reactions. This fact is further illustrated with reference to FIG. 1.

A visual run across the four lines in FIG. 1 allows the sequence to be “read”. It is possible to obtain the sequence simply by determining the number of stopping nucleotides incorporated in each cycle (by the magnitude of measured label, e.g. fluorescence), and the number of intervening nucleotides-incorporate in each cycle (again by magnitude of measured label), and lining up the results for each of four runs using each of the four different nucleotides as stopping nucleotide. Preferably, however, the nature (which may mean identity) of the intervening nucleotides in each run is determined, providing degeneracy of information that allows for very rapid and accurate determination of sequence, allowing for errors in measurement of magnitude of label, for example as discussed further herein.

Base-Calling Algorithm II

More sophisticated basecalling algorithms can be implemented using e.g. dynamic programming, least-squares optimization and/or regular expressions to find an optimal sequence in the face of measurement errors. Such algorithms can also make better use of the redundancy of the available information. In other words, instead of using just the measured length between each occurrence of the same nucleotide, such algorithms would find an optimal sequence that minimizes the difference between the expected and observed abundances of each of the three intervening nucleotides.

The inventor has provided a working dynamic programming algorithm that works well in spite of 20-25% noise. It first performs a multiple alignment of the four series of measurements using dynamic programming, minimizing the difference between the expected and observed abundances of each of the three intervening nucleotides at each step. Then, least squares optimization is used to find the most likely length of each homopolymer stretch based on the four available distance measurements.

Terms and Definitions

A homopolymer is an uninterrupted sequence of one particular nucleotide. A homopolymer sequence is a DNA sequence where homopolymers are written as numbers instead of as repeated letters, i.e., ACCGGT is written ACGT and has homopolymer lengths 1,2,2,1.

Let the chroma be a set of measurements obtained by repeating a method of the invention, such as scheme I, four times, using each of the four natural nucleotides as stopping nucleotides. The chroma thus is a three-dimensional array of measurements indexed by the cycle, the stopping nucleotide and the measured nucleotide. For example, if ten cycles are performed for each stopping nucleotide, the chroma will contain ten (for the number of cycles) times four (for the number of stopping nucleotides) times four (for the number of measured nucleotides) numbers, and the number at location {4, ‘A’, ‘C’} will be the measured fluorescence for cytosine when adenosine was used as stopping nucleotide in cycle number four. For convenience, let chroma for x be the subset of the complete chroma that contains measurements obtained with x as the stopping nucleotide. Thus, the chroma for A is one-fourth of the full chroma.

Let N be the number of cycles performed in each repetition. The chroma therefore is 4*4*N numbers derived from label measurements.

Let a called sequence be a sequence of nucleotides S₀, S₁, . . . Sₖ (where each S is one of [A,C,G,T]). The goal of basecalling is to find an optimal called sequence given the chroma. For convenience, we represent homopolymeric stretches as a quantity instead of by repetition of the same base; in other words, we associate with each position i in the called sequence a quantity qᵢ which gives the estimated number of repetitions of the base Sᵢ. To be consistent, we constrain the sequence such that Sₙ₊₁≈Sₙ for all n.

Basecalling Phase I, Dynamic Programming

The goal of basecalling is to find an optimal called sequence given the chroma sequence. However, there are 4*3^{k-1} possible called sequences of length k, a very large number even for fairly small k (with k=20, there are more than four billion possible called sequences). In order to find a useful basecalling algorithm the complexity of the problem is reduced.

Called sequences can be classified by the number of occurrences of each nucleotide. For example, base counts {1, 2,0,4} correspond to any called sequence containing 1 A, 2 Cs, no Gs and 4 Ts. One example of such a sequence is TCTATCT.

An algorithm provided in accordance with the present invention exploits the fact that we can easily derive the most optimal called sequence in some simple cases, and that more difficult cases can be derived from simpler ones by recursion.

Some simple cases are easy to solve. Base counts {0,0,0,0} corresponds to an empty called sequence. Counts {1,0,0,0} can only correspond to the called sequence ‘A’, and similarly for C, G and T.

However, base counts {1,1,1,1} can correspond to ‘ACGT’, ‘TCGA’ and many others. In such cases the chroma may be used to find the most optimal called sequence.

Note that any called sequence with base counts {i,j,k,l} must correspond exactly to a particular subset of the chroma, namely the subset that includes i cycles of the chroma for A, j cycles of the chroma for C, k cycles of of the chroma for G and l cycles of the chroma for T. Hence a predicted chroma for a called sequence can be compared with the actual measured chroma. The optimal called sequence for {i,j,k,l} would be the one whose predicted chroma was most similar to the relevant subset of the actual measured chroma. Similarity can be measured in many ways, for example as a sum of differences, a sum of square differences, a Pearson correlation coefficient etc. The similarity can be reported as a score, i.e. as an error score to be minimized or a similarity score to be maximized.

The general case {i,j,k,l} cannot be solved directly. But the optimal called sequence for {i,j,k,l} can be generated from shorter sequences in at most four different ways: by adding an ‘A’ to the optimal sequence for {i-1,j,k,l}, by adding a ‘C’ to the optimal sequence for {i j-1,k,l}, by adding a ‘G’ to the optimal sequence for {i,j,k-1,l} or by adding a ‘T’ to the optimal sequence for {i,j,k,l-1}.

One can find out which of the (at most) four extensions is the optimal one by computing a score (as above, by comparing the predicted chroma to the actual) and choosing the minimum (or maximum, depending on the measure used). It is shown below how this can be done, but assume for now that such a score has been found.

We set q for the newly called base to the actual measured quantity obtained from the chroma. For instance, when considering an extension with ‘A’ (i.e. from {-1,j,k,l} to {i,j,k,l}), then q would be obtained from the chroma at location {i, ‘A’, ‘A’}, i.e. the measured quantity of labeled adenosine in cycle i when adenosine was used as stopping nucleotide.

Thus, an optimal called sequence for {i,j,k,l} can always be found by finding the optimal extension of sequences that contain one less of one of the called bases. The procedure may then be repeated for each of the shorter cases, until trivial cases such as {1,0,0,0} are reached. It is therefore always possible to find an optimal called sequence of any length by recursively applying the same simple procedure. As a by-product, the homopolymer lengths qᵢ as measured in the chroma are obtained.

A few restrictions apply:
A sequence cannot contain fewer than zero of any base. Thus we cannot find an optimal called sequence for {i,j,k,0} by extending {i,j,k,-1} with a ‘T’. Because of this restriction, all recursions must ultimately end at {0,0,0,0}, the empty sequence.
Our constraint on called sequences, that Sₙ₊₁≈Sₙ for all n, implies that if the optimal called sequence for {i-1,j,k,l} ends in ‘A’, then we cannot extend with an ‘A’, and so on for the other bases.
In some cases, no extension may be possible. For example, {2,0,0,0} cannot be generated by extension of {1,0,0,0} with another ‘A’. In such cases, no called sequence exists.

The similarity score can be computed in a stepwise manner. Because they differ only by one cycle, the score for {i-1,j,k,l} can be re-used when computing the score for {i,j,k,l}, etc. This may be achieved by keeping track of the length of the optimal called sequence for each {i,j,k,l} as well as the running score. When examining a possible extension from, say, {i-1,j,k,l} to {i,j,k,l} (i.e. extension by an ‘A’), it is only needed to compute the part of the predicted chroma that corresponds to the extra cycle for ‘A’. This may be computed by examining intervening bases in the called sequence back to the most recent ‘A’. Since the optimal called sequence for {i-1,j,k,l} is known it is also known how it was obtained. In particular, the measured quantities q are known for each intervening nucleotide. These are added up for each of ‘C’, ‘G’ and ‘T’ all the way back to the most recent ‘A’ to obtain a prediction for the missing cycle in the predicted chroma. The difference (or square difference etc.) between these predictions and the corresponding cycle in the actual measured chroma are then added to the running score. A normalized score may then be obtained by computing the running score divided by the called sequence length.

Note now, that to compute the optimal called sequence for {3,2,2,2} it is still needed to compute the score for {2,2,2,2}, {1,2,2,2} etc. But in order to find the overall best sequence one must systematically examine all possibilities up to some limit (for example, {N,N,N,N}), each of which will cause recalculation of scores back to {0,0,0,0}, so the combinatorial explosion remains. However, dynamic programming is a clever way of avoiding such combinatorial explosions.

An algorithm may be used so that whenever a score has been computed, it is stored for re-use in a four-dimensional N-by-N-by-N-by-N matrix. Thus when the optimal called sequence for {3,2,2,2} is computed the score for {2,2,2,2}, {1,2,2,2} etc. will be stored in the matrix. When the score for, say, {2,2,2,2} is later needed again, recursion can be avoided altogether and the precomputed result just fetched from the matrix. This provides for a very efficient implementation. Instead of examining something like 3^{4N} possible called sequences, only N⁴ possibilities need to be examined. In a practical system with N=20, for example, the problem is reduced from about 10³⁸ computations to 160 000, changing the algorithm from infeasible to efficient.

The longest sequence that can be confidently called by the algorithm as disclosed here is one that has N homopolymers of one of the bases, more than N of one base and less than N of the others. This is evident from the fact that when N is exceeded in one stopping base, the sequence can still be called because the missing base must go in the holes left by the three others. But when N is exceeded in a second base, the holes left by the remaining bases cannot be unambiguously filled. The limit is not absolute; partial sequence can still be obtained from the entire chroma.

Depending on the application, one may choose to report (among others) the optimal sequence for any {i,j,k,l} up to {N,N,N,N}, the optimal sequence for {N,N,N,N} or the optimal sequence among those where one index is N. In the example below, the latter was used. The choice depends on factors such as if read length is preferred to accuracy and whether partial sequences are acceptable.

Basecalling Phase II, Least Squares (Optional)

The result of phase I is a called sequence S₀, S₁, . . . Sₙ and the corresponding homopolymer lengths q₀, q₁, . . . qₙ. We could write this out in conventional form by rounding each q to the nearest integer and spelling out the resulting DNA sequence. However, there is more information in the chroma that we can make use of to find better estimations for the qᵢ's. After all, the measured homopolymer length of each stopping base is a single measurement, but each position in the called sequence has actually been measured four times (once for each stopping base).

An example makes this clear. Consider the sequence:

ACGCATCAAAGCCTTACACGGTAAGCATCATC

The ‘AAA’ triplet that occurs at position 8 in the sequence will be measured directly in the third step of the chroma for A and will be an approximate number such as 3.43. If the error of measurement is large, it may be difficult to be confident in every case of how to round the measured quantity to an integer.

However, the ‘AAA’ triplet contributes also to the fourth step of the chroma for C, the second step of the chroma for G and the second step of the chroma for T. In two cases (the chromas for C and T) the triplet is actually measured alone, while in the third case it is measured together with the preceding single A. Let’s say the relevant measurements were 3.43, 3.1, 4.2 and 2.9, respectively for the A, C, G and T chromas. We would like to make use of these additional measurements to reduce the effect of random measurement error.

Consider the homopolymer lengths q₀, q₁, . . . qₙ again. Instead of accepting the single numbers obtained in phase I, we can form a set of simultaneous equations that describe additional information about the q's. The triplet above is q₈ since it is the eighth homopolymer. Likewise, the preceding A is q₅. We can now write down the information from the previous paragraph as follows:
q₈=3.43 (from the chroma for A)
q₈=3.1 (from the chroma for C)
q₅+q₈=4.2 (from the chroma for G)
q₈=2.9 (from the chroma for T)

We can proceed in a similar fashion for each position in the called sequence. The resulting system of simultaneous equations can be solved using, e.g., least squares optimization, and the solution gives the set of homopolymer lengths q₀, q₁, . . . qₙ that best matches ALL the measurements in the chroma.

Example of the Error-Tolerant Basecalling Algorithm

The table below shows simulated results of chroma sequencing of the template

ATGGAGCAGCGTCATTCCTTAGCGGGCAACTGTGACGATGGTGAGAAGTC
AGAAAGAGAGGCTCAGGGATTCGAGCATCGGACCTGTATGGACTCTGGGG
A

(the sequenced strand is given) for ten cycles of each stopping nucleotide.

Each block shows the chroma for the indicated stopping nucleotide, each row shows the (simulated) measurements obtained for the nucleotide indicated on the left, in units of one base, and each column is a cycle comprising adding first three then one nucleotide. For example, the four numbers in bold show the measurements obtained in the first cycle of the chroma with DATP as stopping nucleotide. Since the template begins with an A, only A gives a signal significantly different from zero.

| A | | | | | | | | | | |
|---|---|---|---|---|---|---|---|---|---|---|
| A | 0.78 | 1.09 | 1.07 | 1 | 1.03 | 2.01 | 0.86 | 1.17 | 1.03 | 1.99 |
| C | −0.19 | −0.14 | 0.81 | 2.07 | 1.95 | 2.08 | 1.17 | 1.21 | −0.11 | 0.01 |
| G | 0.2 | 2.17 | 1.09 | 1.86 | 0.02 | 3.96 | 1.91 | 1.01 | 3.05 | 0.96 |
| T | 0.07 | 0.86 | 0.03 | 1.31 | 3.57 | −0.14 | 2.19 | 0.09 | 2.1 | 0.08 |
| C | | | | | | | | | | |
| A | 2 | 1.05 | 0.2 | 1.01 | 0.94 | −0.06 | 1.91 | 1.08 | 4.08 | 5.85 |
| C | 0.96 | 1 | 0.98 | 1.95 | 0.92 | 1.04 | 1.1 | 0.99 | 1.05 | 1.14 |
| G | 2.95 | 1.01 | 0.73 | 0.03 | 0.9 | 3.05 | 0.12 | 2.03 | 5.86 | 4.99 |
| T | 1.04 | 0.15 | 0.95 | 2.02 | 1.99 | 0.02 | −0.03 | 2.14 | 3.07 | 0.12 |
| G | | | | | | | | | | |
| A | 0.95 | 1.01 | 1.15 | 0.01 | 2.08 | −0.01 | 2.17 | 0.01 | 1.14 | 1.13 |
| C | 0.06 | 0.02 | 1.01 | 1.11 | 3 | 1.08 | 2.12 | 0.07 | 1.16 | 0.09 |
| G | 2.06 | 0.87 | 1 | 1.06 | 0.97 | 2.98 | 1.08 | 0.92 | 0.99 | 2.02 |
| T | 1.08 | −0.13 | 0.06 | −0.08 | 5.03 | −0.03 | 1.16 | 0.88 | 0.04 | 0.95 |
| T | | | | | | | | | | |
| A | 0.97 | 2.02 | 1.06 | 0 | 3.05 | −0.06 | 1.91 | 0.02 | 2.94 | 6.11 |
| C | −0.07 | 2.01 | 0.81 | 1.91 | 3.16 | 0.06 | 0.9 | 0.07 | −0.1 | 2.24 |
| G | −0.06 | 4.84 | −0.14 | −0.2 | 3.97 | 0.96 | 2.01 | 2.06 | 2.94 | 5.37 |
| T | 1.04 | 0.93 | 2.25 | 2.03 | 1.19 | 0.84 | 0.91 | 0.96 | 0.93 | 0.61 |

Basecalling using the dynamic programming algorithm described above identified the following called sequence (which does not show homopolymers): ATGAGCAGCGTCATCTAGCGCACTGTGACGATG, which is correct. Expanding homopolymers by rounding to the nearest integer yields ATGGAGCAGCGTCATTCCTTAGCGGGCAACTGTGACGATGG, which is again correct, and covers 41 bp of the template. Thus, in only ten cycles of chroma sequencing, and in the presence of significant measurement errors (in this case, 10% CV), one can obtain 41 basepairs of sequence information.

In order to asses the error-tolerance of the given algorithm, a series of one hundred simulations was run on the given template with random noise corresponding to 10% CV. All 100 called sequences and all 100 expanded sequences were correct. 59 of them were 41 bp long, while the rest included an additional T from the template. Thus, the algorithm as presented is both productive and error-tolerant in the face of experimental variance.

Nucleotide Addition Schemes

In SBS it has always been assumed that nucleotides must be added one at a time, or at least must be forced to incorporate one at time as in BASS. However, as shown above, other nucleotide addition schemes can be used to arrive at a DNA sequence, and some are better suited to avoid the limitations of SBS (e.g. loss-of-synchrony). In this section we examine all possible nucleotide addition schemes and show that the regular scheme is in some ways the worst possible.

A nucleotide addition scheme is a rule for adding nucleotides to an SBS reaction. It is comprised of a succession of steps involving the addition of one or more nucleotides. In this section we will ignore any nucleotides added purely as inhibitors or that cannot be incorporated for some other reason. And we will call “T” any nucleotide capable of base-pairing with adenosine (or analogously G, C, A for cytosine, guanine, thymidine). In particular applications, analogs or derivatives of the natural nucleotides may be used, but for sequencing purposes it is their base pairing abilities that determine the logic of a nucleotide addition scheme. Nucleotide analogs or derivatives with multiple base pairing capabilities may be denoted “AC”, “GCT” etc. to indicate this fact.

A cyclic scheme is a nucleotide addition scheme that repeats a basic pattern. A cyclic scheme with restart is a nucleotide addition scheme that repeats a basic pattern and then restarts with fresh primer with a variation of the basic pattern. A natural scheme is one where no base is repeated until all four bases have been added.

Among natural cyclic schemes, “4”, indicating that all four nucleotides are added in the first step, is degenerate and cannot be used for sequencing.

Scheme “1-1-1-1” is the regular scheme, used by all previously disclosed SBS methods. Note that even BASS falls under this category, since although all four nucleotides may be added at the same time, they are forced to incorporate one by one because of a cleavable blocking group.

Scheme 1-1-1-1 is the least productive scheme. This can be seen from the fact that after each productive step, the next nucleotide on the template may be one of three possible (i.e. the three that are different from the base just sequenced), but only a single base is added. As a consequence, it is the scheme most affected by loss of synchrony.

A method according to the present invention is a scheme 3-1, as disclosed herein. It is a fully productive scheme (nucleotides are guaranteed to be incorporated at every step, since the nucleotides absent from a given step are added at the subsequent step). There are four variations of 3-1, given by varying the single nucleotide among A, C, G and T. As shown above, those four variations can be used to reconstruct a target sequence.

Scheme 2-2 is another possible fully productive scheme. There are only three variants of this scheme, corresponding to AC-GT, AG-CT and AT-GC; all other combinations are simple reversals.

What is the minimal requirement for a scheme to ensure that one can always reconstruct the original sequence (possibly with restart). In essence, all that is needed is that each homopolymer in the target sequence must be separable from its two neighbors. In other words, each homopolymer must be part of at least one nucleotide incorporation step that excludes its left-hand neighbor, and one that excludes its right-hand neighbor. In scheme 1-1-1-1, every single step has this property so the sequence can always be reconstructed.

In scheme 3-1, restarting with all four possible variants ensures that each homopolymer is part of a step that includes no other nucleotide. In principle, only three of the four variants are strictly required, since in that case three bases would be added alone in some step, which automatically separates them from the fourth. Thus, scheme 3-1 generates redundant information not present in scheme 1-1-1-1 that can be used to improve basecalling (e.g. through dynamic programming as shown above) in the face of experimental noise. It is thus not only more productive than 1-1-1-1, but also ore error-tolerant.

Scheme 2-2, across three restarts, also generates enough information to call a sequence. It is easy to see that each pair of nucleotides is separable in at least one of AC-GT, AG-CT and AT-GC. Thus scheme 2-2 is possibly the most compact fully productive scheme, although the extra information generated by 3-1 may be worth the effort. Some redundancy is still present (if the nucleotides are labeled with different labels); thus, the error-tolerance of scheme 2-2 is intermediate between 1-1-1-1 and 3-1.

Irregular (non-cyclic) schemes may also be of use in special circumstances. For example, when part of the sequence is known, an irregular scheme might be used to skip over parts that are not of interest faster than would otherwise be possible, or they might be used to generate even more redundant data in order to further reduce basecalling errors.

In conclusion, of the nucleotide addition schemes we have surveyed, 3-1 is the most productive and error-tolerant, while somewhat surprisingly the traditional scheme 1-1-1-1 is the least productive and most error-prone.

Signature Sequencing

Another embodiment of an aspect of the present invention, useful for signature sequencing, comprises a method (scheme III) comprising:

1. Providing a single-stranded template with an annealed primer.

2. Adding three nucleotides, one of which carries a label, e.g. a fluorescent label.

3. Optionally adding one or more nonincorporating inhibitor nucleotides (different from the labeled nucleotides). Examples include 5′-di- and mono-phosphate nucleotides, 5′-(alpha-beta-methylene)triphosphate nucleotides.

4. Incubating with an appropriate polymerase under conditions that cause nucleotides to be added to the growing strand.

5. Detecting the presence and quantity of the labeled nucleotide.

6. Disabling the label, e.g. by photobleaching (not necessarily in every cycle).

7. Adding the remaining nucleotide and incubating with a polymerase (not necessarily the same as in step 5) under conditions that cause nucleotides to be added to the growing strand.

8. Repeating steps 2-7 until the desired number of cycles have been completed.

For example, one may use fluorescent dC and regular dA/dG in step 2 and then add dT in step 7. Step 4 will then add any number of dA, dG and dC until the first occurrence of a dA in the template, then stop because there is no complementary dT nucleotide. The fluorescence read in step 5 will reveal the presence or absence of a dC between each pair of dT. The sequence obtained can in general be written as a binary digit sequence indicating for each successive pair of Ts if there was one or more Cs between them.

For example, the sequence ACGCTACGCATCAGACTC would be written as 1111, and the sequence ACTCAGCTATATT as 11000. In general, such sequences contain information equivalent to ½ basepair per cycle. 24 cycles would be equivalent to a 12 bp signature sequence, and would for example be unique in the human transcriptome. Existing sequence databases and sequence alignment algorithms can readily be adapted to such binary signatures for analysis.

Scheme III is especially easy to implement, as only qualitative measurements are necessary. For example, scheme III may be especially suitable for sequencing single molecules using fluorescence correlation spectroscopy.

Chroma Sequencing using PPi Detection

In another embodiment, an aspect of the present invention provides a method (scheme IV), which comprises (instead of using labeled nucleotides), monitoring the release of inorganic pyrophosphate (PPi) (see e.g. W093/23564). Such a method may comprise:

1. Providing a single-stranded template with an annealed primer.

2. Adding a set of intervening nucleotides (i.e. more than one but less than all of the four possible nucleotides).

3. Optionally adding one or more nonincorporating inhibitor nucleotides (different from the intervening nucleotides). Examples include 5′-di- and mono-phosphate nucleotides, 5′-(alpha-beta-methylene)triphosphate nucleotides.

4. Incubating with an appropriate polymerase under conditions that cause nucleotides to be added to the growing strand, while monitoring the incorporation (e.g. as described in WO93/23564).

5. Adding the set of stopping nucleotides and incubating with a polymerase (not necessarily the same as in step 5) under conditions that cause nucleotides to be added to the growing strand, while monitoring the incorporation (e.g. as described in WO93/23564).

6. Repeating steps 2-5 until the desired number of cycles have been completed.

Again, the scheme can be repeated using each of the four natural nucleotides as stopping nucleotide. Compared to standard pyrosequencing, this protocol provides a four-fold increase in read length with no modifications to the standard protocol (except the change in the order of nucleotide addition and the required changes to basecalling).

The following example shows the significance of loss-of-synchrony and the impact of using the chroma sequencing scheme. It shows the result of a target DNA sequenced with both pyrosequencing and chroma sequencing. It is assumed that a fixed fraction of all templates lose synchrony in each incorporation step. In SBI, steps are additions of a single base. In jump sequencing steps are additions of alternately three or one base. Additionally, chroma sequencing restarts three times with fresh primer, using each of the four natural nucleotides as stopping nucleotide.

The target sequence (the final nucleotide(s) reached by chroma sequencing is shown in capital letter for each stopping nucleotide):

atggagcagc gtcattcctt agcgggcaac tgtgacgatg gtgagaagtc
agaaagagag gctcaGGGat tcgagcatcg gacctgtAtg gactctgggg
atccTTcctt tgggCaaaat gatcccccta ccattttgcc cattactgct

Pyrosequencing

40 stops to loss of synchrony

40 reaction steps

| | |
|---|---|
| Reactions | Results |

| | |
|---|---|
| a c g t | a - - t |
| a c g t | 2g - - - |
| a c g t | a - g - |
| a c g t | - c - - |
| a c g t | a - g - |
| a c g t | - c - - |
| a c g t | - - g t |
| a c g t | - c - - |
| a c g t | a - - 2t |
| a c g t | - 2c - 2t |

total sequence: 20 bp

Chroma Sequencing

40 stops to loss of synchrony

160 reaction steps (i.e. 40 each stopping base)

| | |
|---|---|
| Reactions | Results |

| | | |
|---|---|---|
| cgt a | — | a |
| cgt a | t2g | a |
| cgt a | gc | a |
| cgt a | 2g2ct | a |
| cgt a | 4t2c | a |
| cgt a | . . . etc . . . | |
| cgt a | | |
| cgt a | | |
| cgt a | | |
| cgt a | | |
| cgt a | | |
| cgt a | | |
| cgt a | | |
| cgt a | | |
| cgt a | | |
| cgt a | | |
| cgt a | | |
| cgt a | | |
| cgt a | | |
| cgt a | | |

. . . restart and repeat with [gta c],
[tac g]and [acg t]. . .
total sequence:
88 bp + 27 bp partial sequence

In conclusion, chroma sequencing circumvents the loss-of-synchrony problem, achieving more than four times longer read length.

Solid-Phase Chroma Sequencing

In order to automate and parallelize the method, two main approaches are provided in accordance with embodiments of the present invention.

The first approach uses arrayed or otherwise arranged templates, and is suitable when a large number of templates must be sequenced with retained identity.

The second approach uses random attachment to a solid support and is useful when a large number of sequences must be obtained at random from a library.

A method according to an embodiment of one aspect of the present invention for sequencing arrayed templates provides a method (scheme V) which comprises:

1. Providing a solid support offering a number of active regions or an active surface, each being capable of binding a template molecule
a. directly, or
b. indirectly, by binding a primer or linker that hybridizes or otherwise has affinity with the template.

2. Adding to each active region or to the active surface a single-stranded template, keeping track of which template was placed in each position. Each region would then consist of a large number of identical ssDNA templates, as in spotted microarrays.

3. Optionally adding a primer (or else using the linker from the solid support).

4. Sequencing all templates in parallel in accordance with the invention, e.g. according to any of schemes I-IV.

5. Obtaining for each identified template a sequence. Linkers (step 1b) do not have to be the same in all active regions. Different linkers can be used to fish out particular templates from a complex mixture, providing the possibility of sequencing a subset of a library.

The throughput of scheme V is limited by the resolution of the apparatus used to add template. Densities of several thousand templates per square centimeter are possible using standard microarraying equipment.

When higher throughput is required and template identity is not important, another approach may be used.

A further embodiment of an aspect of the present invention is provided as a method (scheme VI) which comprises:

1. Providing a solid support carrying at least partially single-stranded template molecules attached in random positions (preferably at a density suitable for the detection equipment), each template being optionally amplified to contain multiple copies of the target sequence either attached to or in close proximity to the original template (at least closer than any other template molecule).

2. Sequencing the templates in parallel using the present invention, for example any of schemes I-IV, detecting labeled nucleotides in parallel.

There are many approaches to providing amplified templates at high density. For example, rolling-circle amplification can be used as follows:

a. Provide a surface (e.g. glass) with attached primers, preferably attached via a covalent bond, or, instead of a covalent bond, a very strong non-covalent bond (such as biotin/streptavidin) could be used.

b. Add circular templates, preferably at a density suitable for the detection equipment.

c. Anneal the templates to the primers.

d. Amplify using rolling-circle amplification to produce a long single-stranded tandem-repeated template attached to the surface at each position.

Lizardi et al. describe “Mutation detection and single-molecule counting using isothermal rolling circle amplification”: Nature Genetics vol 19, p. 225.

Modifications to this procedure include providing a reverse primer to generate additional replication forks, increasing product yield. Alternative methods to RCA include solid-phase PCR (Adessi et al. “Solid phase DNA Amplification: characterization of primer attachment and amplification mechanisms” Nucleic Acids Research 2000: 28(20): 87e) and in-gel PCR (‘polonies’, U.S. Pat. No. 6,485,944 and Mitra R D, Church G M, “In situ localized amplification and contact replication of many individual DNA molecules”, Nucleic Acids Research 1999: 27(24):e34).

A “suitable density” is preferably one that maximizes throughput, e.g. a limiting dilution that ensures that as many as possible of the detectors (or pixels in a detector) detect a single template molecule. On any regular array, a perfect limiting dilution will make 37% of all positions hold a single template (because of the form of the Poisson distribution); the rest will hold none or more than one.

For example, on a Typhoon 9200 with a 25 μm pixel size, the 35×43 cm reaction chamber holds 240 million pixels. With a limiting dilution (Poisson distribution), 37% of those would hold a single template, i.e. 89 million templates. Sequencing 50 bases on each template yields 1.7 Gb of sequence in 50 cycles. With a scan time of 45 minutes, daily throughput is about 3 Gbp, equivalent to the full sequence of the human genome.

Templates suitable for solid-phase RCA should optimize the yield (in terms of number of copies of the template sequence) while providing sequences appropriate for downstream applications. In general, small templates are preferable. In particular, templates can consist of a 20-25 bp primer binding sequence and a 40-150 bp insert. The primer binding sequence could be used both to initiate RCA and to prime the sequencing reaction, or the template could contain a separate sequencing primer binding site. The insert should be as small as possible while remaining long enough to contain the desired sequence. For example, if ten cycles of sequencing are performed using a single stopping nucleotide, on average forty bases will be probed and thus the template must at least be longer than forty bases by a comfortable margin to prevent sequencing the primer binding sequence.

In order to increase the signal generated from rolling-circle amplified templates it may be necessary to condense them. Since an RCA product is essentially a single-stranded DNA molecule consisting of as many as 1000 or even 10000 tandem replicas of the original circular template, the molecule will be very long. For example, a 100 bp template amplified 1000 times using RCA would be on the order of 30 μm, and would thus spread its signal across several different pixels (assuming 5 μm pixel resolution). Using lower-resolution instruments may not be helpful, since the thin ssDNA product occupies only a very small portion of the area of a 30 μm pixel and may therefore not be detectable. Thus, it is desirable to be able to condense the signal into a smaller area.

In (Lizardi et al, cited above) the RCA product is condensed by using epitope-labeled nucleotides and a multivalent antibody as crosslinker. In a further aspect, the present invention provides a simple alternative that is especially convenient when sequencing originally double-stranded DNA.

For template preparation for use in a method according to the present invention, and as a further aspect of the invention, dsDNA templates, which may be short e.g. 80 bp, are ligated to linker oligonucleotides carrying hairpin loops to form a pseudo-double stranded, looped structure or a dumbbell shape. In such a structure, primer binding sites for both RCA and the subsequent sequencing reaction can be placed in the hairpin loops. In order to avoid sequencing both strands simultaneously, one can ensure that only templates which have different hairpin loops at their two ends will be sequenced by using different primers for amplification by RCA and for sequencing. Thus, only templates which have at least one RCA primer binding site will be amplified, and only those which have at least one sequencing primer binding site will be sequenced.

Since the RCA product of such a template will be everywhere partially double-stranded, it will fold back into a zig-zag structure that condenses into a smaller area. But since the primer binding sites are everywhere exposed as single-stranded DNA, primer access is not a problem. The example below shows that such templates form ˜5-10 μm products after RCA.

In order to immobilise oligonucleotides to a surface, many different approaches have been described (see e.g., Lindroos et al. “Minisequencing on oligonucleotide arrays: comparison of immobilisation chemistries”, Nucleic Acids Research 2001: 29(13) e69). For example, biotinylated oligos can be attached to streptavidin-coated arrays; NH₂-modified oligos can be covalently attached to epoxy silane-derivatized or isothiocyanate-coated glass slides, succinylated oligos can be coupled to aminophenyl- or aminopropyl-derived glass by peptide bonds, and disulfide-modified oligos can be immobilised on mercaptosilanised glass by a thiol/disulfide exchange reaction. Many more have been described in the literature.

An Apparatus for Automated High-Throughput Sequencing

Methods according to the present invention are particularly suitable for automation, since they can be performed simply by cycling a number of reagent solutions through a reaction chamber placed on or in a detector, optionally with thermal control.

In one example, the detector is a fluorescence scanner, which may for example be operating by laser excitation, bandpass filtering and photomultiplier tube detection. For instance, the ScanArray Express (PerkinElmer) is such an instrument; it scans microscope slides with a resolution of 5 μm/pixel, is capable of detecting as little as 2 fluorochromes per pixel and has a scan time of 20 minutes (in four colors). Daily sequencing throughput on such an instrument would be up to 1.7 Gbp.

The reaction chamber provides:
easy access for the scan head.
a closed reaction chamber.
an inlet for injecting and removing reagents from the reaction chamber.
an outlet to allow air and reagents to enter and exit the chamber.

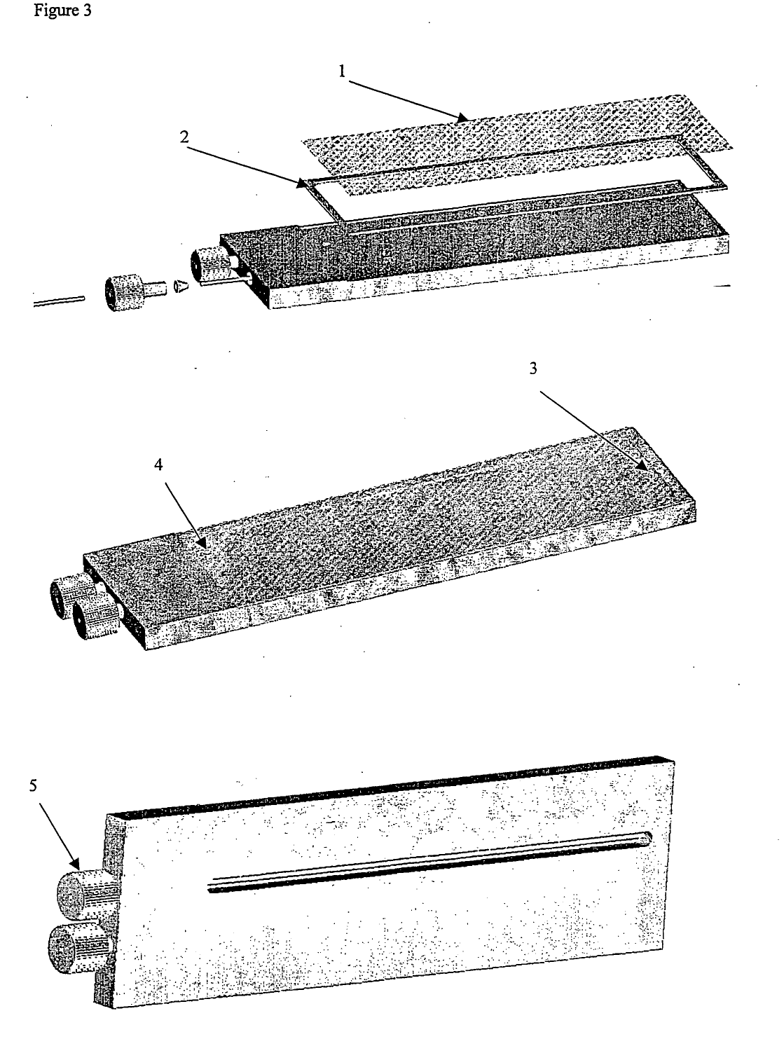

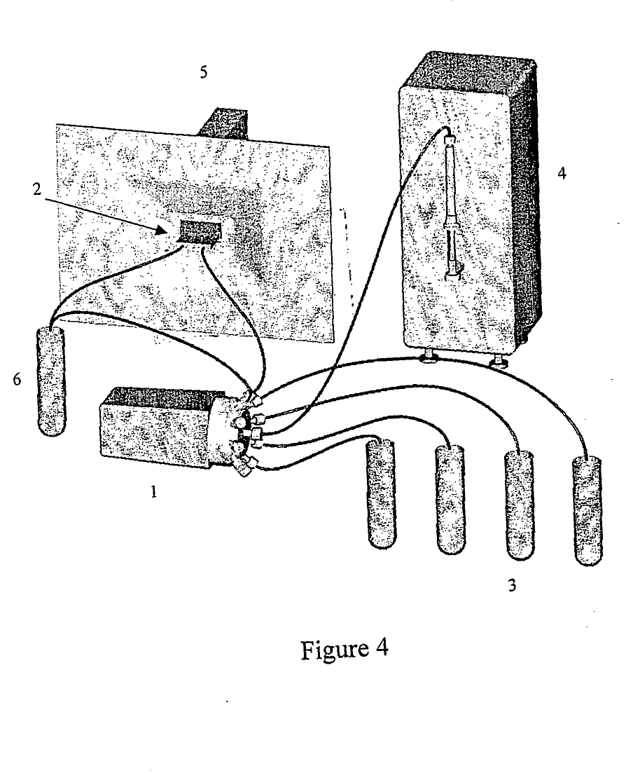

A reaction chamber can be constructed in standard microarray slide format as shown in FIG. 3, suitable for being inserted in a standard microarray scanner such as the ScanArray Express. The reaction chamber can be inserted into the scanner and remain there during the entire sequencing reaction. A pump and reagent flasks (for example as shown in FIG. 4) supply reagents according to a fixed protocol and a computer controls both the pump and the scanner, alternating between reaction and scanning. Optionally, the reaction chamber may be temperature-controlled.

A dispenser unit may be connected to a motorized vent to direct the flow of reagents, the whole system being run under the control of a computer. An integrated system would consist of the scanner, the dispenser, the vents and reservoirs and the controlling computer.

In accordance with a further aspect of the invention there is provided an instrument for performing a method of the invention, the instrument comprising:
an imaging component able to detect an incorporated or released label,
a reaction chamber for holding one or more attached templates such that they are accessible to the imaging component at least once per set of steps,
a reagent distribution system for providing reagents to the reaction chamber.

The reaction chamber may provide, and the imaging component may be able to resolve, attached templates at a density of at least 100/cm², optionally at least 1000/cm², at least 10000/cm² or at least 100 000/cm².

The imaging component may employ a system or device selected from the group consisting of photomultiplier tubes, photodiodes, charge-coupled devices, CMOS imaging chips, near-field scanning microscopes, far-field confocal microscopes, wide-field epi-illumination microscopes and total internal reflection miscroscopes.

The imaging component may detect fluorescent labels.

The imaging component may detect laser-induced fluorescence.

In one embodiment of an instrument according to the present invention, the reaction chamber is a closed structure comprising a transparent surface, a lid, and ports for attaching the reaction chamber to the reagent distribution system, the transparent surface holds template molecules on its inner surface and the imaging component is able to image through the transparent surface.

### EXAMPLE I

### in Situ Template Amplification

A circular single-stranded template was prepared by annealing two 5′-phosphorylated oligonucleotides (TGGTCATCAGCCTTCATGCAACCAAAGTATGAAATAACCAGCGTAATACGACTCACTATAGGGCGTGGTTATTTCATACT and TTGGTTGCATGAAGGCTGATGACCATCCTTTTCCTTACTAGCGTAATACGACTCACTATAGGGCGTAGTAAGGAAAAGGA) at 100 μmol/μl in 4 μl and adding 2 μl T4 ligation buffer, 0.3 μl T4

DNA ligase (1.5 Weiss units; Fermentas) and 7 μl water and incubating at 37 degrees for one hour. The ligase was then inactivated by incubation at 65 degrees for ten minutes.

Primer A50T7RC

(AAAAAAAAAAAAAAAAAAAAAAAAAAAAAAAAAAAAAAAAAAAAAAAAAACGCCCTATAGTGAGTCGTATTACGC), carrying a 5′ terminal amino (—NH) moiety was attached to a Greiner silylated microarray slide by incubating 10 μM primer in 100 μl MOPS (0.2M with sodium acetate and EDTA prepared according to Sambrook et al. ‘Molecular Cloning’, third edition, Cold Spring Harbor Laboratory Press 2001) for 5 minutes, reduced in 1 ml PBS/ethanol (3:1) with 2.5 mg NaBH₄ for 5 minutes and then rinsed in 0.2% sodium dodecyl sulfate followed by distilled water.

Dried slides were then incubated for rolling-circle amplification with 2 μl dUTP-Cy3 (100 μM final, PerkinElmer), 2 μl each of dTTP, DATP, dCTP and dGTP (all 1 mM final, NEB), 4 μl Sequenase buffer, 1 μl Sequenase (13 u, Amersham Biosciences), 4 μl water and 1 μl template. The labeled nucleotides were thus about 2.5% of all nucleotides. After incubation at 37 degrees for two hours, the slide was rinsed in water and scanned on a PerkinElmer ScanArray Express. The result was a large number of bright spots each representing amplified template. The results also show that a labelling frequency of 2.5% can readily be detected in this format (in fact, many spots saturate the detector).

A magnification of a portion of the slide showed that, with a pixel size in the image of 5 μm, most amplified templates occupied one or a small number of pixels. At this size, a very large proportion of the pixels on the scanner could be used for different template molecules, thus ensuring maximal throughput. White pixels completely saturate the detector, showing that at less than 2.5% labelling is more than enough to be detectable. Given that the template was 160 bp, 2.5% labelling represents about 4 incorporated nucleotides per template copy, in the range expected for chroma sequencing reactions.

### EXAMPLE II

### Single Step Sequencing Reaction

Biotinylated T7 primer (GCGTAATACGACTCACTATAGGGCG) was attached to a Greiner streptavidin-coated microarrays slide by incubating in Dynal bind/wash buffer (Dynal, Norway) at 10 μmol/μl. Wells were created on the slide by gluing on a rubber film containing an array of 5 mm wide holes. TOPO2.1 plasmid (Clontech) was boiled, cooled on ice, then added to each well at 20 μmol/μl. After incubating at room temperature for 15 minutes, the slide was washed in bind/wash for 15 minutes.

A reaction mixture containing 4 μl EcoPol buffer, 0.4 μl each of DATP, dTTP and dGTP (100 μM final, NEB), 0.4 μl dUTP-Cy3 (10 μM final, PerkinElmer), 2 μl Klenow exo-DNA polymerase (NEB) and water to 40 μl was added to two wells and an identical mixture replacing Klenow with water was added to two more wells. After incubating for 10 minutes and washing twice for 15 minutes in bind/wash, the slide was scanned on a Typhoon 9200.

Given the template (Clontech TOPO2.1), the expected outcome is 2 dTTP incorporated. FIG. 2 shows the result, clearly indicating that labeled dTTPs were incorporated and that the signal obtained was significantly above background (as given by the fluorescence in the reactions omitting Klenow).

1. A method of determining sequence and/or base composition information for a nucleic acid, the method comprising:
(i) providing a nucleic acid comprising a first strand that comprises a nucleic acid template, wherein a free 3′ end of a nucleic acid strand annealed to the first strand allows for elongation of a strand of nucleic acid complementary to the nucleic acid template by template sequence-dependent incorporation of nucleotides into the strand of nucleic acid complementary to the nucleic acid template by a template-dependent nucleic acid polymerase;
(ii) performing a set of one or more steps, which set of one or more steps is cycled a desired number of times or performed in combination with other sets of one or more steps to elongate the strand of nucleic acid complementary to the nucleic acid template allowing for information indicative of base composition or sequence of the nucleic acid to be obtained,
wherein a step comprises:
(a) providing, in the presence of:
the nucleic acid comprising a first strand that comprises a nucleic acid template,
said free 3′ end of a nucleic acid strand annealed to the first strand of the nucleic acid template, and
a template-dependent nucleic acid polymerase; nucleotides selected from one, two, three or four nucleotide complementarity classes for template-dependent incorporation by the nucleic acid polymerase of the nucleotides into the strand of nucleic acid complementary to the nucleic acid template, wherein each of said nucleotides is a natural nucleotide or a nucleotide analog capable of template-dependent incorporation by a nucleic acid polymerase into a nucleic acid strand at a free 3′ end of the nucleic acid strand, and within each said nucleotide complementarity class the nucleotides and nucleotide analogs are complementary to one of Adenosine (A), Cytosine (C), Thymine (T) and Guanine (G);
and
(b) removing or inactivating unincorporated nucleotides;
and
wherein within a set of steps
nucleotides selected from all four nucleotide complementarity classes are provided and available for template-dependent incorporation,
in at least one step nucleotides selected from more than one, optionally two, three or four, nucleotide complementarity classes are provided and available for template-dependent incorporation, and the nucleotides in at least one of the nucleotide complementarity classes, if incorporated into the strand of nucleic acid complementary to the nucleic acid template, allow further elongation of the strand of nucleic acid complementary to the nucleic acid template, and
optionally no nucleotide complementarity class is provided in more than one step;
and
wherein if nucleotides selected from all four complementarity classes are provided in one step then the nucleotides in one, two or three of the nucleotide complementarity classes, if incorporated into the strand of nucleic acid complementary to the nucleic acid template, prevent further elongation of the strand of nucleic acid complementary to the nucleic acid template and all copies present if multiple copies are present;
(iii) performing multiple sets of said steps, cycling sets of steps and/or performing sets of steps in combination with different sets of steps;
(iv) determining the nature of and/or quantity of nucleotides incorporated into the strand of nucleic acid complementary to the nucleic acid template in at least one set of steps by determining the nature and/or quantity of nucleotides incorporated into the strand of nucleic acid complementary to the nucleic acid template in at least one step in each set for which the nature and/or quantity of nucleotides incorporated is determined for the set.

2. A method according to claim 1 wherein within a set of steps nucleotides selected from three or two of the nucleotide complementarity classes are provided in a first step and nucleotides taken from the remaining one or two nucleotide complementarity classes are provided in a second step.

3. A method according to claim 2 comprising determining the quantity of the nucleotide or nucleotides incorporated in the first or second step in sets of steps for which the nature and/or quantity of nucleotides incorporated is determined.

4. A method according to claim 3 comprising determining the quantity of nucleotides incorporated in each step in sets for which the quantity of nucleotides incorporated is determined.

5. A method according to claim 4 wherein within a set of steps three nucleotides are provided in a first step and one nucleotide is provided in a second step.

6. A method according to claim 5 comprising determining the nature and quantity of nucleotides incorporated in the first step.

7. A method according to claim 2 wherein the nucleotides provided in the first step are labeled, each differently.

8. A method according to claim 2 wherein a nucleotide provided in the second step is labeled.

9. A method according to claim 1 wherein the four nucleotides complementary to A, C, T and G are labeled, each differently.

10. A method according to claim 7, wherein a nucleotide is labeled fluorescently.

11. A method according to claim 7 wherein a label of a nucleotide is disabled when the nucleotide is incorporated into the strand of nucleic acid complementary to the nucleic acid template.

12. A method according to claim 7 wherein a label of a nucleotide is cleaved or released from the nucleotide when the nucleotide is incorporated into the strand of nucleic acid complementary to the nucleic acid template.

13. A method according to claim 12 comprising determining nature and/or quantity of label cleaved or released from one or more nucleotides incorporated into the strand of nucleic acid complementary to the nucleic acid template.

14. A method according to claim 5 comprising performing a cycle of sets of steps wherein within each set of steps in the cycle three nucleotides are provided in a first step and one nucleotide is provided in a second step.

15. A method according to claim 14 comprising performing four cycles of sets of steps for said nucleic acid, wherein within each of the cycles the one nucleotide provided in all the second steps of all the sets of steps is the same, and wherein the one nucleotide provided in all the second steps of all the sets of steps in each cycle is different from the one nucleotide provided in all the second steps of all the sets of steps in the other three cycles.

16. A method according to claim 1, wherein a set of steps additionally comprises providing one or more blocked nucleotides that stop incorporation of nucleotides into the strand of nucleic acid complementary to the nucleic acid template.

17. A method according to claim 1, wherein a set of steps additionally comprises providing one or more non-incorporating inhibitor nucleotides which inhibit misincorporation of nucleotides into the strand of nucleic acid complementary to the nucleic acid template.

18. A method according to claim 1 wherein the nucleic acid template is a deoxyribonucleic acid (DNA), the nucleic acid polymerase is a DNA-dependent DNA polymerase and the nucleotides are deoxyribonucleotides or deoxyribonucleotide analogs.

19. A method according to claim 1 wherein the nucleic acid template is a deoxyribonucleic acid (DNA), the nucleic acid polymerase is a DNA-dependent ribonucleic acid (RNA) polymerase and the nucleotides are ribonucleotides or ribonucleotide analogs.

20. A method according to claim 1 wherein the nucleic acid template is a ribonucleic acid (RNA), the nucleic acid polymerase is a reverse transcriptase and the nucleotides are deoxyribonucleotides or deoxyribonucleotide analogs.

21. A method according to claim 1 wherein the nucleic acid template is provided in multiple copies.

22. A method according to claim 21 comprising providing multiple copies of the nucleic acid template by a nucleic acid amplification reaction.

23. A method according to claim 22 wherein the nucleic acid amplification reaction comprises rolling circle amplification.

24. A method according to claim 23 comprising:
providing a DNA molecule consisting of a stem portion and first and second loop portions, wherein the stem portion consists of a first strand and a second strand, wherein the first strand and second strand are equal in length, complementary and annealed together and comprise a region for which sequence and/or base composition information is desired, wherein the first loop portion joins the 3′ end of the first strand to the 5′ end of the second strand and the second loop portion joins the 3′ end of the second strand to the 5′ end of the first strand so the DNA molecule has no free 5′ or 3′ ends, wherein a loop portion comprise a primer binding site for rolling-circle amplification and a loop portion comprises a primer binding site for sequencing;
performing rolling circle amplification to provide multiple copies of the nucleic acid to serve as said nucleic acid template.

25. A method according to claim 1 wherein the nucleic acid template is attached to a solid support.

26. A method according to claim 25 wherein multiple different nucleic acid templates are attached to a solid support in an array.

27. A method according to claim 25 wherein the nucleic acid template is attached to the solid support via annealing to a primer that is attached to the solid support.

28. A method according to claim 1 comprising determining the sequence of a nucleic acid by analysis of determination of nature and/or quantity of nucleotides incorporated into the strand of nucleic acid complementary to the nucleic acid template.

29. A nucleic acid sequencing-by-synthesis method characterized by incorporation of nucleotides in a step-wise manner, wherein a step allows for template-dependent incorporation of more than one different nucleotide.

30. A method according to claim 29 wherein a step allows for template-dependent incorporation of three different nucleotides selected from the group consisting of nucleotides complementary to Adenosine (A), Cytosine (C), Thymine (T) and Guanine (G), and a separate step allows for template-dependent incorporation of the remaining nucleotide of the group.

31. A computer processor programmed to control a method of according to claim 1.

32. A computer-readable device carrying a program for a computer processor according to claim 31.

33. A computer processor programmed to provide sequence and/or base composition information for a nucleic acid from performance of a method according to claim 1.

34. A computer-readable device carrying a program for a computer processor according to claim 33.

35. A reagent kit suitable for performing a method according to claim 1, the reagent kit including one or more sets of premixed reagents in one or more reagent vessels, wherein each set of premixed reagents comprises
nucleotides taken from all four complementarity classes,
at least one vessel containing nucleotides taken from more than one, optionally two, three or four, complementarity classes, and the nucleotides in at least one of the complementarity classes, if incorporated into the strand of nucleic acid complementary to a nucleic acid template, allow further elongation of the strand of nucleic acid complementary to the nucleic acid template, and
wherein if nucleotides taken from all four complementarity classes are provided in a single vessel then the nucleotides in one, two or three of the of the complementarity classes, if incorporated into the strand of nucleic acid complementary to the nucleic acid template, prevent further elongation of the strand of nucleic acid complementary to the nucleic acid template.

36. An instrument for performing a method according to claim 1, comprising:
an imaging component able to detect an incorporated or released label,
a reaction chamber for holding one or more attached templates such that they are accessible to the imaging component at least once per set of steps,
a reagent distribution system for providing reagents to the reaction chamber.

37. An instrument according to claim 36 wherein the reaction chamber provides, and the imaging component is able to resolve, attached templates at a density of at least 100/cm², optionally at least 1000/cm², at least 10000/cm² or at least 100000/cm².

38. An instrument according to claim 35 wherein the imaging component employs a system or device selected from the group consisting of photomultiplier tubes, photodiodes, charge-coupled devices, CMOS imaging chips, near-field scanning microscopes, far-field confocal microscopes, wide-field epi-illumination microscopes and total internal reflection miscroscopes.

39. An instrument according to claim 35 wherein the imaging component detects fluorescent labels.

40. An instrument according to claim 39 wherein the imaging component detects laser-induced fluorescence.

41. An instrument according to claim 35 wherein the reaction chamber is a closed structure comprising a transparent surface, a lid, and ports for attaching the reaction chamber to the reagent distribution system, where the transparent surface holds template molecules on its inner surface and the imaging component is able to image through the transparent surface.

42. A DNA molecule consisting of a stem portion and first and second loop portions, wherein the stem portion consists of a first strand and a second strand, wherein the first strand and second strand are equal in length, complementary and annealed together, wherein the first loop portion joins the 3′ end of the first strand to the 5′ end of the second strand and the second loop portion joins the 3′ end of the second strand to the 5′ end of the first strand so the DNA molecule has no free 5′ or 3′ ends.

43. A DNA molecule according to claim 42 wherein a loop portion comprises a primer binding site for rolling-circle amplification.

44. A DNA molecule according to claim 42 wherein a loop portion comprises a primer binding site for sequencing.

45. An array of multiple different DNA molecules according to claim 42, attached to a solid support, optionally via annealing to primers attached to the solid support.

46. A method of making a DNA molecule according to claim 42 the method comprising:
providing a double-stranded DNA molecule consisting of
a first strand which has a 5′ end and a 3′ end, and
a second strand which has a 5′ end and a 3′ end; and
ligating a first linker to join the 3′ end of the first strand to the 5′ end of the second strand, and ligating a second linker to join the 3′ end of the second strand to the 5′ end of the first strand, wherein the linkers are hairpin structures.

47. A method of producing multiple copies of a DNA template, the method comprising performing rolling-circle amplification on a DNA molecule according to claim 43 to produce an elongated DNA molecule comprising multiple copies of the DNA template.

48. A method of producing multiple copies of multiple DNA templates, the method comprising performing rolling-circle amplification on multiple DNA molecules according to claim 43 to produce multiple elongated DNA molecules comprising multiple copies of the DNA templates.

49. A method according to claim 47 wherein a rolling circle amplification primer or the DNA molecules are attached to a solid support.

50. A method according to claim 47 further comprising condensing the elongated DNA molecules by annealing between complementary strands within the multiple copies of the DNA template within the elongated DNA molecules.

51. A method according to claim 50 wherein the elongated DNA molecules are condensed onto a solid support.

52. A method according to claim 47 further comprising sequencing multiple copies of the DNA template or DNA templates within the elongated DNA molecules.
